(19) United States
(12) Patent Application Publication
(10) Pub. No.: US 2021/0177487 A1
(43) Pub. Date: Jun. 17, 2021

(54) MEDICAL HEATER, TREATMENT TOOL, AND TREATMENT TOOL MANUFACTURING METHOD

(71) Applicant: OLYMPUS CORPORATION, Tokyo (JP)

(72) Inventor: Tsunetaka AKAGANE, Hachioji-shi (JP)

(73) Assignee: OLYMPUS CORPORATION, Tokyo (JP)

(21) Appl. No.: 17/185,307

(22) Filed: Feb. 25, 2021

Related U.S. Application Data
(63) Continuation of application No. PCT/JP2018/033619, filed on Sep. 11, 2018.

(51) Int. Cl. A61B 18/08; H01B 1/02; H01B 7/04; H05B 3/20; H05B 3/12

(52) U.S. Cl. CPC A61B 18/085; H01B 1/02; A61B 2018/00095; H05B 3/20; H05B 3/12; H01B 7/04

(57) ABSTRACT

A medical heater includes: a substrate having a first plate surface and a second plate surface; and a conductive portion provided on the first plate surface. The substrate is folded back in a state where the first plate surface forms an outer surface in a longitudinal direction of the substrate, the conductive portion includes: a pair of connecting portions to which wiring members are electrically connected; a heat generating portion; and an electric path portion that is connected from the connecting portions to the heat generating portion, and the heat generating portion has a configuration in which a resistance value of the heat generating portion is higher than resistance values of other parts in the conductive portion, and a thickness measurement of at least a part of the heat generating portion is smaller than thickness measurements of other parts in the conductive portion.

### CROSS-REFERENCE TO RELATED APPLICATION

This application is a continuation of International Application No. PCT/JP2018/033619, filed on Sep. 11, 2018, the entire contents of which are incorporated herein by reference.

### BACKGROUND

### 1. Technical Field

The present disclosure relates to a medical heater, a treatment tool, and a treatment tool manufacturing method.

### 2. Related Art

There is a known treatment tool that applies energy to a site as a target of treatment (hereinafter, referred to as a target site) in a biological tissue for treatment of the target site (refer to US 2015/0327909 A, for example).

The treatment tool described in US 2015/0327909 A includes a pair of gripping members to grip the target site. The gripping member includes a medical heater that generates heat when energized, and a treatment member that comes into contact with the target site when the target site is gripped by the pair of gripping members. The treatment tool allows the heat from the medical heater to be transferred to the target site gripped with the pair of gripping members via the treatment member. This achieves treatment of the target site.

Furthermore, a medical heater described in US 2015/0327909 A includes a substrate and a conductive portion provided on the substrate. The conductive portion includes first and second connecting portions to which individual wiring members are electrically connected, and a heat generating portion that generates heat when energized. The first and second connecting portions are disposed side by side in a width direction of the substrate on the proximal end side of the substrate. Furthermore, the heat generating portion has a substantially U-shape extending from the proximal end side toward the distal end side, folded back on the distal end side, and extending back to the proximal end side on the substrate. In addition, either end of the heat generating portion is electrically connected to the first and second connecting portions, individually. That is, the conductive portion has two electric paths parallel to each other in the width direction of the substrate.

### SUMMARY

In some embodiments, a medical heater includes: a substrate having a first plate surface and a second plate surface forming front and back surfaces of the substrate, the substrate being electrically insulating and flexible; and a conductive portion provided on the first plate surface. The substrate is folded back in a state where the first plate surface forms an outer surface in a longitudinal direction of the substrate, the conductive portion includes: a pair of connecting portions to which wiring members are electrically connected, each connecting portion being provided at either end of the substrate in the longitudinal direction; a heat generating portion configured to generate heat when energized; and an electric path portion that is connected from the connecting portions to the heat generating portion so as to energize the heat generating portion, and the heat generating portion has a configuration in which a resistance value of the heat generating portion is higher than resistance values of other parts in the conductive portion, and a thickness measurement of at least a part of the heat generating portion is smaller than thickness measurements of other parts in the conductive portion.

In some embodiments, a treatment tool includes: a treatment member having a treatment surface on which treatment of a biological tissue is performed and an installation surface forming front and back surfaces of the treatment member with the treatment surface; and a medical heater configured to heat the treatment member. The medical heater includes: a substrate having a first plate surface and a second plate surface forming front and back surfaces of the substrate, the substrate being electrically insulating and flexible; and a conductive portion provided on the first plate surface, the substrate is folded back in a longitudinal direction of the substrate in a state where the first plate surface forms an outer surface of the substrate, the conductive portion includes: a pair of connecting portions to which wiring members are electrically connected, each connecting portion being provided at either end of the substrate in the longitudinal direction; a heat generating portion configured to generate heat when energized; and an electric path portion that is connected from the connecting portions to the heat generating portion so as to energize the heat generating portion, the heat generating portion has a configuration in which a resistance value of the heat generating portion is higher than resistance values of other parts in the conductive portion, and a thickness measurement of at least a part of the heat generating portion is smaller than thickness measurements of other parts in the conductive portion, and the medical heater is installed in a state where the heat generating portion faces the installation surface.

In some embodiments, a treatment tool manufacturing method includes: forming a conductive portion including a heat generating portion, on a first plate surface of a substrate; folding back the substrate in a longitudinal direction of the substrate in a state where the first plate surface forms an outer surface of the substrate so as to form a medical heater; and installing the medical heater on a treatment member having an installation surface on which treatment of a biological tissue is performed in a state where the heat generating portion faces the installation surface. The conductive portion includes: a pair of connecting portions to which wiring members are electrically connected, each connecting portion being provided at either end of the substrate in the longitudinal direction; the heat generating portion configured to generate heat when energized; and an electric path portion that is connected from the connecting portions to the heat generating portion so as to energize the heat generating portion, and formation of the conductive portion is performed in a state where a thickness measurement of at least a part of the heat generating portion is smaller than thickness measurements of other portions in the conductive portion.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a view illustrating a treatment system according to a first exemplary embodiment;

FIG. 2 is a view illustrating a gripping portion;

FIG. 3 is a view illustrating a gripping portion;

FIG. 4 is a view illustrating a medical heater;

FIG. 5 is a view illustrating a medical heater;

FIG. 6 is a view illustrating a medical heater;

FIG. 7 is a flowchart illustrating a method of manufacturing a treatment tool;

FIGS. 8A to 8D are views illustrating a method of manufacturing a treatment tool;

FIG. 9 is a view illustrating a method of manufacturing a treatment tool; and

FIG. 10 is a view illustrating a medical heater according to a second exemplary embodiment.

### DETAILED DESCRIPTION

Hereinafter, modes for carrying out the disclosure (hereinafter referred to as embodiments) will be described with reference to the drawings. The disclosure is not limited to the embodiments described below. In the drawings, same reference signs are attached to the same components.

Schematic Configuration of Treatment System

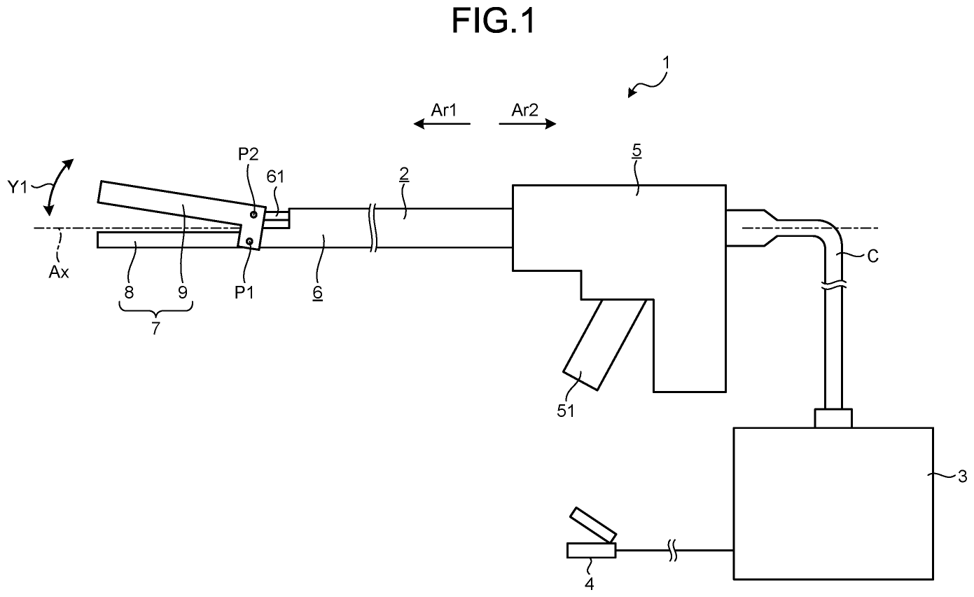

FIG. 1 is a view illustrating a treatment system 1 according to a first exemplary embodiment.

The treatment system 1 applies thermal energy to a site as a treatment target (hereinafter, referred to as a target site) in a biological tissue, and thereby achieves treatment of the target site. Here, the treatment typically includes coagulation and incision of the target site. As illustrated in FIG. 1, the treatment system 1 includes a treatment tool 2, a control device 3, and a foot switch 4.

Configuration of Treatment Tool

The treatment tool 2 is a surgical treatment tool for performing the treatment of a target site through the abdominal wall, for example. As illustrated in FIG. 1, the treatment tool 2 includes a handle 5, a shaft 6, and a gripping portion 7.

The handle 5 is a part held by a surgeon. As illustrated in FIG. 1, the handle 5 includes an operation knob 51.

The shaft 6 has a substantially cylindrical shape. In the following, one side running along a central axis Ax of the shaft 6 will be referred to as a distal end side Ar1 (FIG. 1), while the other side will be referred to as a proximal end side Ar2 (FIG. 1). A part of the proximal end side Ar2 of the shaft 6 is inserted into the handle 5 from the distal end side An of the handle 5, whereby the shaft 6 is attached to the handle 5. In addition, the shaft 6 internally includes a movable member 61 (FIG. 1) that reciprocates along the central axis Ax in accordance with the operation of the operation knob 51 by the surgeon. Furthermore, an electric cable C (FIG. 1) has one end connected to the control device 3 and the other end provided through the inside of the handle 5 and the shaft 6 to reach the gripping portion 7.

Configuration of Gripping Portion

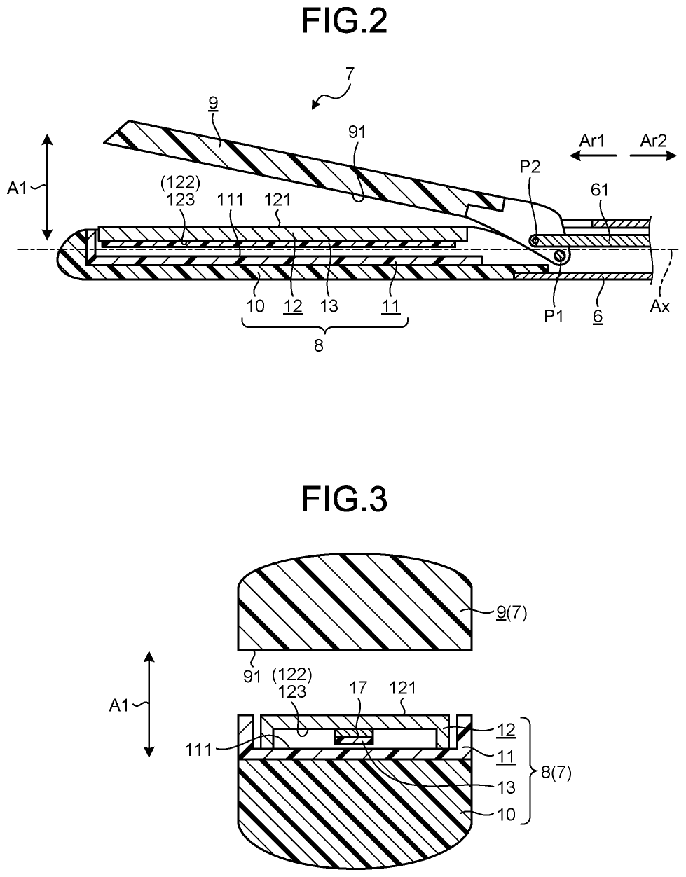

FIGS. 2 and 3 are views illustrating the gripping portion 7. Specifically, FIG. 2 is a cross-sectional view of the gripping portion 7 cut along a plane along the central axis Ax. FIG. 3 is a cross-sectional view of the gripping portion 7 cut by a plane orthogonal to the central axis Ax.

The gripping portion 7 is a portion that is used for treatment of the target site while gripping the target site. As illustrated in FIGS. 1 to 3, the gripping portion 7 includes first and second gripping members 8 and 9.

The first and second gripping members 8 and 9 are configured to be openable/closable in a direction of arrow Y1 (FIG. 1) in accordance with the operation of the operation knob 51 by the surgeon.

Configuration of First Gripping Member

In FIGS. 2 and 3, the first gripping member 8 is arranged on the lower side with respect to the second gripping member 9. As illustrated in FIG. 2 or 3, the first gripping member 8 includes a support member 10, a heat insulating member 11, a treatment member 12, and a medical heater 13.

The support member 10 has an elongated shape extending in a longitudinal direction (left-right direction (direction along the central axis Ax) in FIG. 2) connecting the distal end and the proximal end of the gripping portion 7, with one end of the support member 10 being fixed to an end of the distal end side Ar1 of the shaft 6. In addition, the support member 10 uses its upper surface in FIGS. 2 and 3 to support the heat insulating member 11, the treatment member 12, and the medical heater 13.

Examples of the material constituting the support member 10 described above include a metal material such as stainless steel or titanium.

The heat insulating member 11 has an elongated shape extending in the longitudinal direction of the gripping portion 7, and is fixed to the upper surface of the support member 10 in FIGS. 2 and 3.

There is provided a recess 111 on the upper surface of the heat insulating member 11 in FIGS. 2 and 3, extending from the proximal end of the heat insulating member 11 toward the distal end side Ar1. The heat insulating member 11 supports the treatment member 12 and the medical heater 13 in the recess 111.

Examples of the material constituting the heat insulating member 11 described above include a resin material having a low thermal conductivity such as polyetheretherketone (PEEK). That is, by arranging the heat insulating member 11 having a low thermal conductivity between the treatment member 12, the medical heater 13, and the support member 10, it is possible to efficiently transfer the heat from the medical heater 13 to the treatment member 12.

The treatment member 12 has an elongated shape extending in the longitudinal direction of the gripping portion 7 and is fixed in the recess 111.

The upper surface of the treatment member 12 in FIGS. 2 and 3 comes into contact with the target site while the target site is gripped by the first and second gripping members 8 and 9. That is, the upper surface functions as a treatment surface 121 (FIGS. 2 and 3) that applies thermal energy to the target site. In addition, “application of thermal energy to the target site” means transfer of the heat from the medical heater 13 to the target site. In the first embodiment, the treatment surface 121 is formed with a flat surface orthogonal to mutually opposing directions A1 (FIGS. 2 and 3) in the first and second gripping members 8 and 9 in a case where the first and second gripping members 8 and 9 are set to closed states of gripping the target site.

Although the first embodiment is an example in which the treatment surface 121 is formed of a flat surface, the treatment surface 121 is not limited to this and may be formed of other shapes such as a protruding shape or a recessed shape. The same applies to a gripping surface 91 described below.

Furthermore, on an installation surface 122 of the treatment member 12, there is a recess 123 (FIGS. 2 and 3) formed to extend from the proximal end to the distal end of the treatment member 12. The installation surface 122 forms front and back surfaces of the treatment member 12 with the treatment surface 121. The treatment member 12 supports the medical heater 13 by the bottom surface of the recess 123.

Examples of the material constituting the treatment member 12 described above include materials with high thermal conductivity, such as copper, silver, aluminum, molybdenum, tungsten, graphite, or a composite material of these.

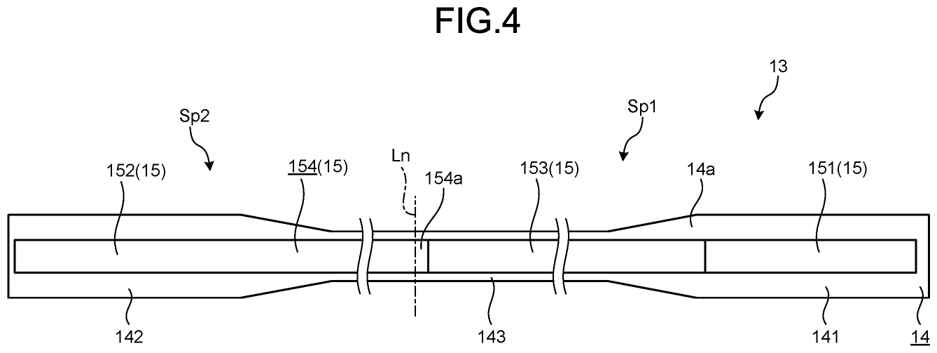

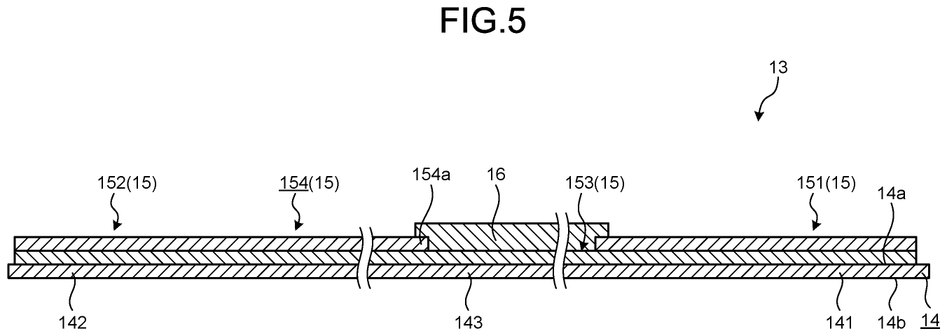

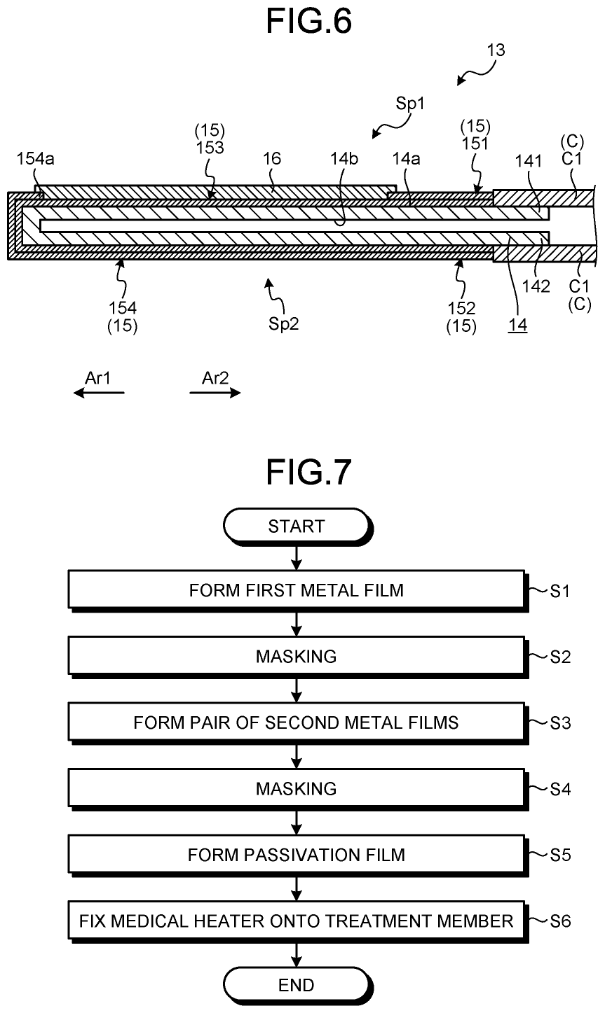

FIGS. 4 to 6 are views illustrating the medical heater 13. Specifically, FIG. 4 is a view of the medical heater 13 in a state before a substrate 14 is folded back, as viewed from a first plate surface 14a side of the substrate 14. FIG. 5 is a cross-sectional view of the medical heater 13 in a state before the substrate 14 is folded back, cut by a plane orthogonal to the width direction (left-right direction in FIG. 3) of the substrate 14. FIG. 6 is a cross-sectional view of the medical heater 13 in a state after the substrate 14 is folded back, cut along a plane orthogonal to the width direction of the substrate 14.

The medical heater 13 is a sheet-type heater that partially generates heat when energized. As illustrated in FIGS. 4 to 6, the medical heater 13 includes the substrate 14, a conductive portion 15, and a passivation film 16 (FIGS. 5 and 6).

The substrate 14 is a sheet-like flexible substrate formed of a resin material having electrical insulation such as polyimide. The substrate 14 has an elongated shape, and includes: first and second wide portions 141 and 142 located at either end in the longitudinal direction (in FIGS. 4 and 5 in the left-right direction); and a narrow portion 143 located between the first and second wide portions 141 and 142 and connecting the first and second wide portions 141 and 142.

Here, the width measurement (length measurement in the up-down direction in FIG. 4) of the narrow portion 143 is set to a substantially uniform measurement in the longitudinal direction. Furthermore, the width measurement in the narrow portion 143 is set smaller than that in the first and second wide portions 141 and 142.

Among the first plate surface 14a (FIGS. 4 to 6) and a second plate surface 14b (FIGS. 5 and 6) forming front and back surfaces of the substrate 14, the conductive portion 15 is provided on the first plate surface 14a. As illustrated in FIGS. 4 to 6, the conductive portion 15 includes first and second connecting portions 151 and 152, a heat generating portion 153, and an electric path portion 154.

The first and second connecting portions 151 and 152 correspond to the connecting portions according to the disclosure. As illustrated in FIG. 4, the first and second connecting portions 151 and 152 are provided on the first and second wide portions 141 and 142, respectively. That is, the first and second connecting portions 151 and 152 are provided at either end in the longitudinal direction of the substrate 14, individually. The first and second connecting portions 151 and 152 are individually electrically connected to a pair of lead wires C1 (FIG. 6) constituting the electric cable C.

The heat generating portion 153 is connected, at one end of the heat generating portion 153, to the first connecting portion 151 and extends, on the other end side of the heat generating portion 153, linearly toward the second connecting portion 152 side.

The electric path portion 154 is a portion provided as an energizing path to the heat generating portion 153, and is connected, at one end side of the electric path portion 154, to the other end of the heat generating portion 153, while extends, on the other end side of the electric path portion 154, linearly toward the second connecting portion 152 side. Here, one end of the electric path portion 154 connected to the heat generating portion 153 corresponds to a heat generating-side end 154a (FIGS. 4 to 6) according to the disclosure. The other end of the electric path portion 154 is connected to the second connecting portion 152. Note that the second connecting portion 152 and the electric path portion 154 may be formed separately or integrally. That is, the electric path portion 154 is connected from the first and second connecting portions 151 and 152 to the heat generating portion 153 and energizes the heat generating portion 153.

As described above, the conductive portion 15 is provided on the first plate surface 14a, in a state of being connected in series in the order of the first connecting portion 151, the heat generating portion 153, the electric path portion 154, and the second connecting portion 152 in the longitudinal direction of the substrate 14.

In addition, with the first and second connecting portions 151 and 152, the heat generating portion 153, and the electric path portion 154 set to have predetermined total lengths and cross-sectional areas, the heat generating portion 153 is set to have a resistance value that is higher than the values in the other parts in the conductive portion 15, namely, the first and second connecting portions 151 and 152, and the electric path portion 154. Therefore, when a voltage is applied to the first and second connecting portions 151 and 152 via the pair of lead wires C1 under the control of the control device 3, the heat generating portion 153 mainly generates heat.

Specifically, in the first embodiment, the width measurements (length measurements in the up-down direction in FIG. 4) of the first and second connecting portions 151 and 152, the heat generating portion 153, and the electric path portion 154 are set to be the same measurements. Here, the width measurement of the heat generating portion 153 is preferably half or more of the width measurement of the narrow portion 143. Furthermore, the thickness measurement of the heat generating portion 153 (length measurement in the up-down direction in FIG. 5) is set smaller than the thickness measurement of the first and second connecting portions 151 and 152 and the electric path portion 154. The thickness measurements of the first and second connecting portions 151 and 152 and the electric path portion 154 are set to be the same.

Furthermore, in the first embodiment, by appropriately setting the material, the total length, and the cross-sectional area, the conductive portion 15 is set to have a resistance value (hereinafter, referred to as a heater resistance) 30 [Ω] to 150 [Ω] in the conductive portion 15 at room temperature. Here, the width measurement of the conductive portion 15 (length measurement in the up-down direction in FIG. 4) and the total length of the conductive portion 15 (length measurement in the left-right direction in FIG. 4) are restricted to some extent in accordance the specifications of the treatment tool 2 (specifications of the gripping portion 7). Therefore, by controlling the material and thickness measurement of the conductive portion 15 (length measurement in the up-down direction in FIG. 5), the heater resistance at room temperature is set to the above-described value. Specifically, examples of the material constituting the conductive portion 15 include a material containing nickel, specifically, stainless steel, nickel, or a nickel alloy. Furthermore, an exemplary range of the thickness measurement of the heat generating portion 153 is several tens [nm] to several [μm].

The passivation film 16 is constituted with nickel fluoride and covers a part of the surface of the conductive portion 15 as illustrated in FIG. 5 or 6. Specifically, the passivation film 16 covers the surface of the heat generating-side end 154a and extends from the surface of the heat generating-side end 154a to the first connecting portion 151 side so as to cover a part of the surface of the first connecting portion 151. That is, the passivation film 16 covers the entire surface of the heat generating portion 153. The passivation film 16 is not limited to the configuration of covering the entire surface of the heat generating portion 153, and may be configured to cover the surface of the heat generating-side end 154a and a part of the surface of the heat generating portion 153.

The medical heater 13 described above is fixed to the bottom surface of the recess 123 by an adhesive sheet 17 (FIG. 3) in a state where the substrate 14 is folded back.

Here, the adhesive sheet 17 is located between the bottom surface of the recess 123 and the medical heater 13 so as to adhere the bottom surface and the medical heater 13. The adhesive sheet 17 is formed by mixing a material having high thermal conductivity, high temperature resistance, and adhesiveness, for example, epoxy resin, with a ceramic having high thermal conductivity, such as alumina and aluminum nitride.

As illustrated in FIG. 6, the substrate 14 is folded back with reference to a folding line Ln (FIG. 4) which is orthogonal to the longitudinal direction of the substrate 14 and is located substantially in the center of the same longitudinal direction in a state where the first plate surface 14a forms an outer surface of the medical heater 13. In other words, the substrate 14 is folded back in a state where the second plate surface 14b is located inside with reference to the folding line Ln. In this state, the first and second wide portions 141 and 142 face each other. The folding line Ln is not limited to a line that is exactly orthogonal to the longitudinal direction of the substrate 14, but also includes a line that intersects the longitudinal direction within a predetermined angle range.

In the following, for convenience of explanation, the region on the first connecting portion 151 side with respect to the folding line Ln will be referred to as a treatment-side region Sp1, and the region on the second connecting portion 152 side with respect to the folding line Ln will be referred to as a back-side region Sp2.

As illustrated in FIG. 4, the electric path portion 154 is provided across the folding line Ln. Therefore, the first connecting portion 151, the heat generating portion 153, and the heat generating-side end 154a are located in the treatment-side region Sp1. Furthermore, the second connecting portion 152 and regions of the electric path portion 154 other than the heat generating-side end 154a are located in the back-side region Sp2.

The substrate 14 is folded back with reference to the folding line Ln as described above and is fixed to the bottom surface of the recess 123 with the adhesive sheet 17 in a state where the treatment-side region Sp1 faces the bottom surface.

Configuration of Second Gripping Member

The second gripping member 9 has an elongated shape extending in the longitudinal direction of the gripping portion 7. In the second gripping member 9, the proximal end side Ar2 is pivotably supported with respect to the shaft 6 about a fulcrum P1 (FIGS. 1 and 2). Furthermore, in the second gripping member 9, the proximal end side Ar2 is pivotably supported with respect to the movable member 61 about a fulcrum P2 (FIGS. 1 and 2). That is, the second gripping member 9 pivots about the fulcrum P1 together with the reciprocation of the movable member 61 along the central axis Ax in accordance with the operation of the surgeon on the operation knob 51. This operation allows the second gripping member 9 to perform open/close operation with respect to the first gripping member 8.

Here, the lower surface in FIG. 2 of the second gripping member 9 functions as the gripping surface 91 to grip the target site, together with the treatment surface 121. In the first embodiment, the gripping surface 91 is formed as a flat surface orthogonal to the directions A1.

The first embodiment has described an exemplary configuration in which the first gripping member 8 (support member 10) is fixed to the shaft 6, and the second gripping member 9 is pivotally supported by the shaft 6. However, the disclosure is not limited to this configuration. For example, it is allowable to adopt a configuration in which both the first and second gripping members 8 and 9 are pivotally supported with respect to the shaft 6, and the first and second gripping members 8 and 9 perform open/close operation by pivot movements individually. Furthermore, for example, it is also allowable to adopt a configuration in which the first gripping member 8 is pivotally supported with respect to the shaft 6 while the second gripping member 9 is fixed to the shaft 6, and the first gripping member 8 performs open and close operations with its pivot movement with respect to the second gripping member 9.

Configuration of Control Device and Foot Switch

The foot switch 4 is a part operated by the surgeon with own foot. Treatment control performed by the control device 3 is executed in accordance with the operation on the foot switch 4.

Note that, the device used for execution of the treatment control is not limited to the foot switch 4, and other devices such as manual operation switches or the like may be employed.

The control device 3 includes a central processing unit (CPU) or the like, and executes treatment control of controlling the treatment tool 2 to operate in accordance with a predetermined program, thereby performing treatment of a target site.

Operation of Treatment System

Next, operations of the treatment system 1 described above will be described.

The surgeon holds the treatment tool 2 by hand and inserts the distal end (a part of the gripping portion 7 and the shaft 6) of the treatment tool 2 into the abdominal cavity through the abdominal wall using a trocar, for example. The surgeon also operates the operation knob 51. The surgeon grips the target site by the gripping portion 7. Thereafter, the surgeon operates the foot switch 4. Subsequently, the control device 3 executes the following treatment control.

The control device 3 applies a voltage to the first and second connecting portions 151 and 152 via the pair of lead wires C1. Here, the control device 3 measures the heater resistance based on the voltage value and the current value supplied to the conductive portion 15 by using a voltage drop test method, for example. Furthermore, the control device 3 refers to resistance temperature characteristics measured in advance. The resistance temperature characteristics are characteristics indicating a relationship between the heater resistance and the temperature of the heat generating portion 153 (hereinafter referred to as the heater temperature). The control device 3 controls the heater resistance to a target resistance value corresponding to the target temperature in the resistance temperature characteristics while changing the electric power supplied to the conductive portion 15. With this control, the heater temperature is controlled to the target temperature. That is, the heat from the heat generating portion 153 controlled to the target temperature is transferred to the target site through the treatment member 12.

The treatment control described above makes it possible to achieve incision with coagulation in the target site.

Treatment Tool Manufacturing Method

Next, a method for manufacturing the above-described treatment tool 2 will be described.

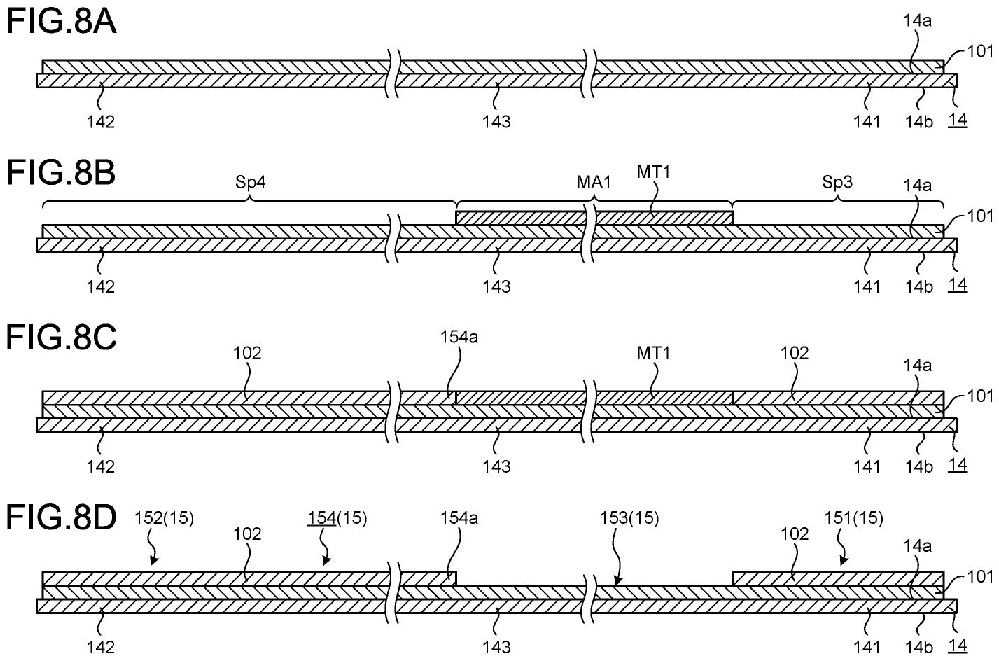

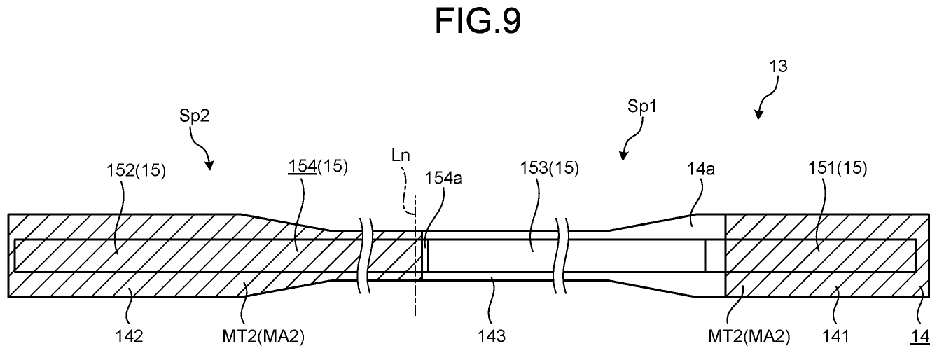

FIG. 7 is a flowchart illustrating a method of manufacturing a treatment tool 2. FIGS. 8A to 8D and FIG. 9 are views illustrating a method of manufacturing the treatment tool 2. Specifically, FIGS. 8A to 8D are views that correspond to FIG. 5. FIG. 9 is a view that corresponds to FIG. 4.

First, as illustrated in FIG. 8A, an operator performs electroless plating to form a first metal film 101 extending in the longitudinal direction of the substrate 14 on the first plate surface 14a of the substrate 14 (step S1). The first metal film 101 is constituted with a material containing nickel, specifically, stainless steel, nickel, or nickel alloy.

After step S1, the operator uses masking tape MT1 (FIG. 8B) to mask a second region MA1 (FIG. 8B) between first regions Sp3 and Sp4 (FIG. 8B) on the first metal film 101 spaced apart from each other in the longitudinal direction of the substrate 14 (step S2).

After step S2, as illustrated in FIG. 8C, the operator uses electroplating to form a pair of second metal films 102 onto the first regions Sp3 and Sp4 on the first metal film 101 (step S3). Thereafter, the operator removes the masking tape MT1 as illustrated in FIG. 8D.

The first and second metal films 101 and 102 are constituted as the conductive portion 15 as illustrated in FIG. 8D. Furthermore, the second region MA1 on the first metal film 101 is constituted as the heat generating portion 153. Furthermore, the first regions Sp3 and Sp4 and the pair of second metal films 102 on the first metal film 101 are constituted as the first and second connecting portions 151 and 152 and the electric path portion 154, respectively. The second connecting portion 152 and the electric path portion 154 may be formed separately or integrally as described above.

After step S3, the operator uses masking tape MT2 (FIG. 9) to mask the regions excluding the region where the passivation film 16 is provided, specifically in the present embodiment, regions excluding the surface of the heat generating portion 153 and the surface of the heat generating-side end 154a (step S4). In FIG. 9, for convenience of explanation, a third region MA2 masked by the masking tape MT2 is represented by hatched lines.

After step S4, the operator places the substrate 14 in an atmosphere of a gas containing fluorine and performs heating at a predetermined temperature so as to perform surface modification of the region on the surface of the conductive portion 15 other than the masked third region MA2 (step S5). With this process, as illustrated in FIG. 5, the passivation film 16 constituted with nickel fluoride is formed in the regions other than the masked third region MA2, that is, on the surface of the heat generating portion 153 and the surface of the heat generating-side end 154a. Thereafter, the operator removes the masking tape MT2.

Note that in a case of forming the passivation film 16 on a part of the surface of the heat generating portion 153 and the surface of the heat generating-side end 154a, it is only needed to mask regions excluding a part of the surface of the heat generating portion 153 and the surface of the heat generating-side end 154a.

After step S5, as illustrated in FIG. 6, the operator folds back the substrate 14 in a state where the first plate surface 14a constitutes the outer surface with reference to the folding line Ln so as to achieve formation of the medical heater 13. Furthermore, with a posture in which the folding line Ln is located on the distal end side Ar1 and in a state where the treatment-side region Sp1 faces the bottom surface of the recess 123, the operator fixes the medical heater 13 onto the bottom surface by the adhesive sheet 17 (step S6).

According to the first embodiment described above, the following effects are obtained.

In the medical heater 13 according to the first embodiment, the conductive portion 15 is provided on the first plate surface 14a, in a state of being connected in series in the order of the first connecting portion 151, the heat generating portion 153, the electric path portion 154, and the second connecting portion 152 in the longitudinal direction of the substrate 14. Furthermore, the substrate 14 is folded back with reference to the folding line Ln in a state where the first plate surface 14a constitutes the outer surface of the medical heater 13.

That is, the substrate 14 having electrical insulation is present between the treatment-side region Sp1 in the conductive portion 15 and the back-side region Sp2 in the conductive portion 15. This makes it possible to prevent an occurrence of short circuit between the treatment-side region Sp1 in the conductive portion 15 and the back-side region Sp2 in the conductive portion 15.

Furthermore, the conductive portion 15 has a configuration extending in the longitudinal direction (left-right direction in FIG. 4) of the substrate 14. The substrate 14 is folded back with reference to the folding line Ln, thereby allowing the treatment-side region Sp1 in the conductive portion 15 and the back-side region Sp2 in the conductive portion 15 to be arranged in parallel to each other in the directions A1. That is, there is no need to arrange the two electric paths in parallel in the width direction of the substrate 14, making it possible to reduce the width measurement of the substrate 14.

Meanwhile, in the medical heater described in US 2015/0327909 A, the heat generating portion has a shape extending while meandering in a wavy shape in order to increase the resistance value of the heat generating portion. That is, the known technique has a reduced width measurement of the heat generating portion with the elongated total length of the heat generating portion. In such a configuration, when the heat generating portion is covered with the adhesive sheet, a void might be formed between the peaks or between valleys of the wavy shape in the heat generating portion. Heating the heat generating portion with the void might produce a state in which the heat is trapped in the void, causing overheating in the part of the heat generating portion in proximity to the void, leading to disconnection of the part.

Fortunately, however, in the medical heater 13 according to the first embodiment, the thickness measurement of the heat generating portion 153 is smaller than that of the first and second connecting portions 151 and 152 and the electric path portion 154. That is, it is possible to reduce the cross-sectional area of the heat generating portion 153, eliminating the need to have a wavy shape of the heat generating portion as described in US 2015/0327909 A, enabling the width measurement of the heat generating portion 153 to be set to the large width measurement same as the measurements of the first and second connecting portions 151 and 152 and the electric path portion 154. Therefore, by achieving the setting of large width measurement of the heat generating portion 153, it is possible to avoid disconnection of the heat generating portion 153.

Furthermore, in the medical heater 13 according to the first embodiment, the heat generating portion 153 is constituted with a material containing nickel.

Furthermore, the surface of the heat generating portion 153 is covered with the passivation film 16 constituted with nickel fluoride.

Here, it is assumed a case where in the course of the use of the treatment tool 2, a part of the medical heater 13 has been removed from the bottom surface of the recess 123, leading to a state where a part of the treatment-side region Sp1 on the first plate surface 14a is exposed in the recess 123. Even in this case, since the surface of the heat generating portion 153 is covered with the passivation film 16, it is possible to suppress the corrosion or oxidation of the heat generating portion 153 or occurrence of rusting on the heat generating portion 153 that would cause a change in the resistance temperature characteristics measured in advance. That is, even when the treatment tool 2 is used for a long period of time, the heater temperature can be controlled to the target temperature by using the resistance temperature characteristics measured in advance.

In particular, the heat generating portion 153 is constituted with a material containing nickel. The passivation film 16 is constituted with nickel fluoride.

Therefore, by exposing the surface of the heat generating portion 153 to an atmosphere containing fluorine, the passivation film 16 is formed by surface modification of the heat generating portion 153. That is, there is no need to provide a special device using a chemical vapor deposition or the like in the formation of the passivation film 16, making it possible to reduce the manufacturing cost of the medical heater 13. Furthermore, since the passivation film 16 is formed by surface modification of the heat generating portion 153, the passivation film 16 can be a dense film, and this enables an extremely small thickness measurement of the passivation film 16. Therefore, the passivation film 16 would not deteriorate the thermal conductivity from the heat generating portion 153 to the treatment member 12. That is, the treatment performance of the target site would not deteriorate.

Furthermore, in the medical heater 13 according to the first embodiment, the electric path portion 154 is provided across the folding line Ln. That is, in the state where the substrate 14 is folded back with reference to the folding line Ln, the electric path portion 154 is folded back. Here, the electric path portion 154 has a larger thickness measurement than the heat generating portion 153. Therefore, as compared with the case where the heat generating portion 153 is folded back, it is possible to suppress the disconnection of the conductive portion 15, and thus possible to sufficiently ensure the durability of the conductive portion 15.

Furthermore, in the medical heater 13 according to the first embodiment, the passivation film 16 covers not merely the surface of the heat generating portion 153 but also the surface of the heat generating-side end 154a of the electric path portion 154. Here, the heat generating-side end 154a is a portion connected to the heat generating portion 153, and thus, likely to have a high temperature. That is, in the course of use of the treatment tool 2, corrosion or oxidation of the heat generating-side end 154a and rusting at the heat generating-side end 154a are likely to occur.

Therefore, by covering the surface of the heat generating-side end 154a with the passivation film 16, it is possible to suppress corrosion or oxidation of the heat generating-side end 154a or occurrence of rusting on the heat generating-side end 154a that can cause a change in the resistance temperature characteristics measured in advance. That is, even when the treatment tool 2 is used for a long period of time, the heater temperature can be controlled to the target temperature by using the resistance temperature characteristics measured in advance.

Furthermore, in the first embodiment, formation of the conductive portion 15 is performed by forming the first metal film 101 on the first plate surface 14a by electroless plating (step S1), and by forming the pair of second metal films 102 on the first metal film 101 by electroplating (step S3).

This facilitates formation of the heat generating portion 153, the first and second connecting portions 151 and 152, and the electric path portion 154 having different thickness measurements from each other.

### Second Embodiment

Next, a second embodiment will be described.

In the following description, identical reference numerals are given to the components similar to those in the first embodiment described above, and detailed description thereof will be omitted or simplified.

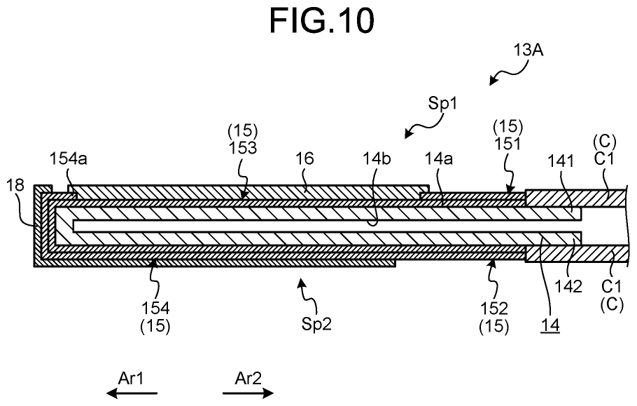

FIG. 10 is a view illustrating a medical heater 13A according to the second embodiment. Specifically, FIG. 10 is a view that corresponds to FIG. 6.

As illustrated in FIG. 10, the medical heater 13A according to the second embodiment has a difference in that a cover member 18 is added, compared with the medical heater 13 in the first embodiment described above.

The cover member 18 is provided across the folding line Ln on the first plate surface 14a of the substrate 14. Specifically, the cover member 18 extends from a position at which a predetermined gap is provided toward the second connecting portion 152 side from the passivation film 16, onto the second connecting portion 152 side so as to cover the surface of the electric path portion 154. That is, the cover member 18 covers regions of the electric path portion 154 other than the heat generating-side end 154a.

Examples of the cover member 18 described above include a material having electrical insulation, such as a coverlay film, a sealing material, a melt layer of polyimide.

According to the second embodiment described above, the following effects are obtained in addition to the effects similar to the case of the first embodiment described above.

The medical heater 13A according to the second embodiment includes the cover member 18.

Therefore, with the presence of the cover member 18, it is possible to improve the watertightness of the back-side region Sp2 in the conductive portion 15. Furthermore, since the cover member 18 has electrical insulation, it is possible to prevent an occurrence of a short circuit between the treatment-side region Sp1 in the conductive portion 15 and the back-side region Sp2 in the conductive portion 15 even when a liquid enters the recess 111.

Furthermore, the cover member 18 covers regions of the electric path portion 154 other than the heat generating-side end 154a. That is, since the cover member 18 is provided at a position avoiding the heat generating-side end 154a, which is likely to have a high temperature, there will be no concern about a case where the cover member 18 has a high temperature, making it possible to prevent the removal of the cover member 18 from the first plate surface 14a.

### Other Embodiments

While the above is description of the modes for carrying out the disclosure, the disclosure should not be limited by only the first and second embodiments described above.

Although, in the above-described first and second embodiments, a configuration in which thermal energy is applied to the target site is adopted, the disclosure is not limited to this. It is also allowable to adopt a configuration in which high frequency energy or ultrasonic energy is applied in addition to the thermal energy. Note that, “applying high frequency energy to the target site” means sending a radio frequency current through the target site. Furthermore, “applying ultrasonic energy to the target site” means applying ultrasonic vibration to the target site.

In the above-described first and second embodiments, the medical heaters 13 or 13A according to the disclosure is provided only on the first gripping member 8. However, the disclosure is not limited to this, and the medical heaters 13 or 13A according to the disclosure may be provided on both of the first and second gripping members 8 and 9.

In the above-described first and second embodiments, an example in which a material containing nickel is used as the material constituting the conductive portion 15 is exemplified. However, the disclosure is not limited to this, and another material can be adopted as long as it is any of stainless steel, nickel, nickel alloy, palladium, platinum, gold, and silver, or a combination of these.

In the above-described first and second embodiments, the first and second metal films 101 and 102 are formed by electroless plating and electroplating, respectively. However, the film formation is not limited to this and the films may be formed by sputtering.

According to the medical heater, the treatment tool, and the treatment tool manufacturing method according to the disclosure, it is possible to reduce a width measurement of a substrate, while preventing the short circuit of the conductive portion provided on the substrate.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical heater comprising:
a substrate including:
a first plate surface forming a front surface of the substrate; and
a second plate surface forming a back surface of the substrate, the substrate being electrically insulating and flexible; and
a conductive portion provided on the first plate surface,
wherein the substrate is folded such that the first plate surface forms an outer surface in a longitudinal direction of the substrate,
the conductive portion includes:
a first connecting portion electrically connected to a first wiring member;
a second connecting portion electrically connected to a wiring member, the first connecting portion being provided a first end of the substrate in the longitudinal direction and the second connecting portion being provided at a second end of the substrate in the longitudinal direction;
a heat generating portion configured to generate heat when energized; and
an electric path portion configured to energize the heat generating portion by being connected to the first connecting portion, the second connecting portion and the heat generating portion; and
the heat generating portion having a resistance value that is higher than resistance values of other parts in the conductive portion, and a thickness measurement of a part of the heat generating portion is smaller than thickness measurements of other parts in the conductive portion.

2. The medical heater according to claim 1,
wherein the conductive portion includes at least one material selected from the group consisting of stainless steel, nickel, nickel alloy, palladium, platinum, gold, and silver.

3. The medical heater according to claim 1,
the heat generating portion comprising a material that includes nickel.

4. The medical heater according to claim 3,
wherein a part of a surface of the heat generating portion is covered with a passivation film that includes nickel fluoride.

5. The medical heater according to claim 4,
wherein the electric path portion is provided across a folding line orthogonal to the longitudinal direction of the substrate and includes a material containing nickel, and
the passivation film is configured to cover the part of the surface of the heat generating portion and a surface of a heat generating-side end of the electric path portion, the heat generating-side end being connected to the heat generating portion.

6. The medical heater according to claim 5, further comprising a cover member that includes a material having electrical insulation, the cover member being configured to cover regions of the electric path portion other than the heat generating-side end.

7. The medical heater according to claim 1,
wherein the heat generating portion, the first connecting portion, the second connecting portion, and the electric path portion are same in width measurements orthogonal to the longitudinal direction of the substrate.

8. A treatment tool comprising:
a treatment member including a treatment surface on which treatment of a biological tissue is performed and an installation surface including front and back surfaces of the treatment member with the treatment surface; and
a medical heater configured to heat the treatment member,
wherein the medical heater includes:
a substrate including a first plate surface that is configured to form a front surface of the substrate, and a second plate surface that is configured to form a back surface of the substrate, the substrate being electrically insulating and flexible; and
a conductive portion provided on the first plate surface,
the substrate is folded in a longitudinal direction of the substrate so that the first plate surface forms an outer surface of the substrate,
the conductive portion includes:
a first connecting portion that is connected to a first wiring member;
a second connecting portion that is connected to a second wiring member, the first connecting portion being provided at a first end of the substrate in the longitudinal direction and the second connecting portion being connected to a second end of the substrate in the longitudinal direction;
a heat generating portion configured to generate heat when energized; and
an electric path portion is configured to energize the heat generating portion by being connected to the first connecting portion, the second connecting portion, and the heat generating portion so as to energize the heat generating portion,
the heat generating portion has a resistance value of the heat generating portion that is higher than resistance values of other parts in the conductive portion, and a thickness measurement of at least a part of the heat generating portion is smaller than thickness measurements of other parts in the conductive portion, and
the medical heater is installed such that the heat generating portion faces the installation surface.

9. The treatment tool according to claim 8, further comprising an adhesive sheet that includes a material having electrical insulation, the adhesive sheet being configured to adhere the first plate surface and the installation surface to each other.

10. The treatment tool according to claim 8,
wherein the heat generating portion includes a material containing nickel, and
at least a part of a surface of the heat generating portion is covered with a passivation film that includes nickel fluoride.

11. A treatment tool manufacturing method comprising:
forming a conductive portion including a heat generating portion, on a first plate surface of a substrate;
folding the substrate in a longitudinal direction of the substrate such that the first plate surface forms an outer surface of the substrate so as to form a medical heater; and
installing the medical heater on a treatment member the treatment member including an installation surface on which treatment of a biological tissue is performed such that the heat generating portion faces the installation surface,
wherein the conductive portion includes:
a first connecting portion connected to a first wiring member and a second connecting portion connected to a second wiring member, the first connecting portion provided at a first end of the substrate in the longitudinal direction and the second connecting portion provided at a second end of the substrate in the longitudinal direction;
the heat generating portion configured to generate heat when energized; and
an electric path portion being configured to energize the heat generating portion by being connected to the first connecting portion, the second connecting portion, and
formation of the conductive portion such that a thickness measurement of at least a part of the heat generating portion is smaller than thickness measurements of other portions in the conductive portion.

12. The treatment tool manufacturing method according to claim 11,
wherein the formation of the conductive portion is performed by including:
forming a first metal film extending in the longitudinal direction of the substrate, on the first plate surface;
forming a second metal film on the first metal film;
providing, in the formation of the second metal film, a first region on the first metal film on which the second metal film is formed and a second region on which the second metal film is not formed;
constituting the first connecting portion, the second connecting portion, and the electric path portion by the first metal film on the first region and by the second metal film; and
constituting the heat generating portion by the first metal film on the second region.

13. The treatment tool manufacturing method according to claim 12,
wherein the first metal film includes a material containing nickel, and
at least a part of a surface of the heat generating portion undergoes surface modification in an atmosphere of a gas containing fluorine so as to form a passivation film including nickel fluoride on at least the part of the surface of the heat generating portion.
